(19) United States
(12) Patent Application Publication
(10) Pub. No.: US 2023/0310764 A1
(43) Pub. Date: Oct. 5, 2023

(54) INHALER AND METHOD AND ARRANGEMENT FOR ADMINISTERING AN ACTIVE SUBSTANCE

(71) Applicant: Körber Technologies GMBH, Hamburg (DE)

(72) Inventors: Christian Hanneken, Hamburg (DE); Michael Joseph Capone, Hamburg (DE)

(21) Appl. No.: 18/023,200

(22) PCT Filed: Aug. 25, 2021
(86) PCT No.: PCT/EP2021/073512

§ 371 Date: Feb. 24, 2023

(30) Foreign Application Priority Data
Aug. 31, 2020 (DE) 10 2020 122 651.9

(51) Int. Cl. A61M 15/00; A61M 11/00; A61M 16/00; A61M 11/04

(52) U.S. Cl. CPC A61M 15/0085; A61M 11/005; A61M 16/0003; A61M 11/045; A61M 2016/0018

(57) ABSTRACT

An inhaler that includes a housing having a body configured to receive an active substance container containing an active substance, and an air channel extending within the housing; an administration element for nebulising or vaporising the active substance for mixing the active substance with air flowing in the air channel; an electronic control apparatus; an electronic data storage device; and a sensor system having a flow measuring apparatus for measuring a volume flow and a mass flow of the air and the active substance for storage as active substance delivery data; wherein: the electronic control apparatus is configured to convert active substance administration data into administration data to control the mixing of the active substance with the air; and the electronic control apparatus is configured to calculate a quantity of the active substance delivered from the active substance container and to compare the quantity with the active substance administration data.

### CROSS-REFERENCE TO RELATED APPLICATIONS

This application is the U.S. National Stage of PCT/EP2021/073512 filed on Aug. 25, 2021, which claims priority to German Patent Application 102020122651.9 filed on Aug. 31, 2020, the entire content of both are incorporated herein by reference in their entirety.

### FIELD OF THE INVENTION

The invention relates to an inhaler, comprising a housing having a main body, the main body being configured and adapted to receive an active substance container containing an active substance, an air channel extending within the housing between at least one air inlet opening and one inhalation opening, an administration element for nebulising or vaporising the active substance fed from the active substance container to the administration element for mixing with air flowing in the air channel, an electronic control apparatus, an electronic data storage device and a sensor system having a flow measuring device for measuring the volume flow and/or mass flow of the air flowing through the air channel and/or of the active substance flowing through the air channel for storage as active substance delivery data in the electronic data storage device.

The invention additionally relates to a method for administering an active substance with an inhaler.

The invention also addresses an arrangement configured and adapted for administering an active substance, comprising an inhaler and a communication terminal comprising a mobile application and/or an application terminal and/or cloud storage.

### BACKGROUND OF THE INVENTION

Such inhalers, which can be configured as a single part or multiple parts, are used in the luxury goods/stimulants industry, here in particular in connection with an electronic cigarette, the so-called e-cigarette (also referred to as ENDS=Electronic Nicotine Delivery System), as well as in the pharmaceutical/medical field to enable the inhalation of fluid and solid luxury goods/stimulant products and/or fluid and solid pharmaceutical/medical products or active substances as mist, in vapour form, as an aerosol or as a vapour/aerosol mixture. When consuming tobacco products or administering active substances, a person usually sucks on a mouthpiece of the inhaler which, in an air channel with an air inlet opening and in the region of the mouthpiece with an air outlet side, creates a suction pressure that generates an air flow through the air channel. The mouthpiece has an inhalation opening via which the user brings about an intake of the active substance. The air flow can also be generated mechanically, e.g. by a pump. The active substance is usually delivered to the air channel by an administration element. The administration element is synonymous with all components that provide a solid and/or liquid active substance as an inhalable substance. The administration element, for example, can be a vaporiser unit. The vaporiser unit, for example, can include a heating member and a wick member. However, the vaporiser unit can likewise have a laser light source or the like as the vaporising member. Within the scope of the present invention, devices for generating mechanical propelling forces, compressors or the like for generating inhalable substances are also included in the term administration element or vaporiser unit.

Individual components of the inhaler, namely, for example, a main body, an active substance container and an administration element, can be combined in a common component, e.g. a vaporiser cartridge. The inhaler or individual components thereof can be configured as a disposable item which is designed for a finite number of inhalation puffs by a consumer or a user. The inhaler can also be configured as a reusable item, it being necessary in each case to adapt the components to the different use of the inhaler. Individual components like the active substance container are regularly configured as disposable items.

An electronic control apparatus and an energy source are known for operating and controlling an inhaler. The energy source, for example, can be a disposable electrochemical battery or a rechargeable electrochemical battery, e.g. a lithium-ion battery, by means of which the administration element, the electronic control apparatus, the electronic data storage device or other equipment are supplied with energy via electrical contacts. The electronic and/or electrical control apparatus is used to control the electrical and/or electronic data processing processes occurring in connection with the inhaler.

### SUMMARY OF THE INVENTION

The use of an inhaler is an oral way of administering drugs to a patient, and thus therapeutically or pharmacologically active combinations of active substances. If inhalers are used as part of a therapeutic approach, a physician or other person authorised to provide therapy prescribes a treatment consisting of a drug and a regimen. In the following, the term drug and active substance are to be understood synonymously, as the drug contains the active substance.

Drugs to be administered by inhalers comprise the desired active substance which is stored in a container, for example a canister, and is administered for inhalation by means of the inhaler. Inhalers usually have a spray valve which releases a set quantity of the active substance with each spray. The manufacturer selects the inhaler which releases the desired dosage per spray. The canister is usually a disposable product. The main body of the inhaler, however, is regularly configured for repeated use. The active substance to be administered by the inhaler is usually present in an inhalation medium. The inhalation medium with the active substance is usually stored in an active substance container for this purpose. The inhalation medium is stored on or in the inhaler. Various mixtures with different constituents of the same or different vapour densities are used as the inhalation medium. For use in the pharmaceutical/medical or therapeutic field, e.g. for inhaling systemically or pneumologically effective drugs, the mixture can correspondingly comprise pharmacological constituents and active substances as well as active substance combinations. Typically, an inhalation medium for medical use comprises a solvent in which the active substance is dissolved. Different alcohols, for example, serve as solvents. A typical mixture for use in an e-cigarette, on the other hand, has, for example, constituents of glycerine and propylene glycol, possibly enriched with nicotine and/or almost any other flavourings. The term active substance is therefore understood to mean all substances that can be inhaled for enjoyment and/or therapeutic purposes.

A regimen describes predetermined application periods as well as the course of taking or administering a drug. Sometimes regimens are also referred to as exercise, plan, therapy plan, etc. When determining the regimen, the physician must consider various factors, such as the age, weight, comorbidities and/or medications already prescribed for the patient. It is not possible for the attending physician to check the dose of the active substance actually taken, since the physician cannot usually monitor the intake, which is why the quantity of an active substance actually consumed can differ from the planned quantity. If there is evidence that the patient is not taking enough of the active substance, the physician can increase the dose by modulating the regimen, for example, he can add more periods of time and/or increase the repetitions in which the drug should be taken via the inhaler. The regimen thus specifies the dose or frequency with which the drug is to be taken in order to achieve the best possible effect.

Today's electronic cigarette products and inhalers dispense the active substance to be delivered only via a preset, user-independent administration mechanism, comprising an administration controller and an administration element. In this case, the control system activates the administration element, for example as a result of a negative pressure measured by a sensor while inhalation is taking place. The quantity of active substance delivered in this case is substantially determined by the length of time the administration element is activated and also cannot be modified.

A further disadvantage is that with the inhalers currently available, the efficacy of a treatment and the availability of data about the treatment carried out depends heavily on the self-discipline of the patient. The efficacy can be impaired if the patient does not consistently follow the regimen and/or does not use the inhaler correctly.

In addition, conventional inhalers cannot monitor or record the time and the duration of use. Moreover, the inhalers can be used at any time of the day without restriction and the patient can freely and uncontrollably change the dose. For example, the physician can only evaluate whether the regimen has been effective by means of further checks and make adjustments if necessary. With the current inhalers, there is no way of verifying and/or monitoring whether the drug has been administered according to the proposed regimen. In addition, many factors can result in the regimen not being followed between any follow-up appointments. The patient may not understand or forget the application period and decrease the frequency by mistake or misuse, or may also increase it intentionally. This can lead, for example, to patients taking excessive amounts of active substance, their prescriptions run out prematurely, leaving their conditions untreated.

Furthermore, a conventional inhaler cannot adjust the quantity of active substance released to the patient. The entire dose is usually released as soon as the inhaler is activated. This results in the patient having to inhale deeply at the same time in order to inhale the entire quantity of active substance provided by the inhaler. However, the lung function of patients varies or may fluctuate widely throughout a day. For example, high humidity or exertion can greatly reduce the ability of the patient to absorb the drug. If the patient breathes only a fraction of a second after operating the inhaler, if the patient breathes irregularly, if the patient has shallow breathing or has a small volume of air, the quantity not inhaled remains in the air channel or the mouthpiece, in the chamber of the inhaler or in the oral cavity of the patient. In any case, there is no guarantee that the active substance delivered is absorbed completely. Some active substances can cause fungal growth in the oral cavity if they are incorrectly inhaled. For this purpose, the instructions for use of the inhalers regularly include information according to which it is recommended to rinse the mouth after inhalation. However, if inhalation processes are incorrect, the quantity of active substance taken by the patient is less than the quantity of active substance prescribed by the physician and the success of treatment may be diminished.

A conventional inhaler has no feedback for monitoring the operation performed or taking of the active substance. The patient and attending physician have no way of ensuring that an inhaler is used consistently according to the instructions prescribed. In addition, the patient is not provided with any resources to safely use the inhaler or take the drug. Thus, the physician cannot establish a causal relationship between not taking the drug and the therapeutic success. Without knowing that the patient has forgotten to take the drug several times, the physician will take further action to achieve the desired therapeutic success. Examples of such action are increasing the dosage of the drug or increasing the frequency of taking the drug or using a different active substance.

In currently available inhalers, the container or canister has no functional coupling to the administration element of the inhaler and is not connected to it. The patient can use a wide variety of containers for their inhaler which, for example, administer a larger quantity of the active substance per spray and/or have different concentrations of active substance than the manufacturer or physician have provided or prescribed.

The object of the present invention is to monitor the use of an inhaler and dynamically adjust the administration of an active substance for safe and controllable administration of drugs or an inhalation medium having an active substance to a patient or user and thus to improve the efficacy of treatments.

This object is achieved by an inhaler of the type referred to hereinbefore in that the electronic control apparatus is configured and adapted to convert active substance administration data provided in the electronic data storage device for administration of the active substance by the inhaler into administration data in order to control the mixing, by the administration element, of the active substance with air flowing in the air channel, and the electronic control apparatus being configured to calculate the active substance delivered from the active substance container based on the active substance delivery data detected by means of the flow measuring apparatus and to compare same with the active substance administration data.

With the configuration of the inhaler according to the invention, it is possible to accurately detect the active substance actually taken by the user. In this way, it can reliably be detected whether the quantity of active substance delivered by the administration element matches the quantity of active substance which has flowed through the air channel. The structure of the inhaler thus serves in particular to detect specific data that allow conclusions to be drawn about the quality of the inhalation process. The design according to the invention of the electronic control apparatus for controlling the mixing of the active substance, by the administration element, with the air flowing in the air channel, which is performed in particular by the user when sucking on the air channel, provides a simple way of varying the quantity of active substance to be delivered to the air channel in further inhalation processes. Depending on the quantity of the active substance delivered, it is thus possible, using the configuration of the inhaler, to specifically set the active substance administration data or the administration data based on the active substance delivery data.

The active substance administration data are also known in the medical context as exercise, therapy plan, therapy regimen, etc. The active substance administration data are usually defined by the authorised person (e.g. the physician) and/or the provider of the active substance, i.e. the active substance administration data con be provided directly with the inhaler or the active substance container. Ideally, the user of the inhaler should not be able to make any manual changes to the active substance administration data. When the authorised person (physician) generates the active substance administration data, they can use technical resources (machine processing). The regimen primarily influences the treatment and defines the dose which is determined, among other things, by the active substance used. The inhaler converts the active substance administration data into the administration data which can preferably be adjusted dynamically but always follow the overall specifications of the active substance administration data. The administration data can differ from the active substance administration data, although the parameters of the total dose and the active substance remain the same. When determining the active substance administration data, the physician, for example, must consider various factors, such as the age, weight, comorbidities and/or medications already prescribed for the patient.

Conversion of the active substance administration data into administration data by means of the electronic control apparatus enables the inhaler to be operated reliably. Due to the large number of electronic components in the inhaler, there is not provision for manual operation of the inhaler, in particular the administration element. The active substance administration data include the desired quantity of active substance to be delivered to the user with the inhaler, for example during an inhalation process. The electronic control apparatus converts the active substance administration data into machine-readable information, by means of which the administration element controls the mixing of the active substance in the air channel. The administration data are preferably also stored in the electronic data storage device.

The administration element, or for example also called the active substance dispenser, is activated in particular by means of the electronic control apparatus. One way of controlling the administration element preferably includes controlling negative pressure measured by means of a sensor while inhalation is taking place. The quantity of active substance delivered in this case is substantially determined by the length of time the administration element is activated. In preferred embodiments, an administration element can be a heating element, an ultrasonic nebuliser which vaporises or nebulises liquid using a piezoelectric element, a gas compressor which builds up a gas pressure and thereby nebulises or vaporises liquid through a nozzle, or a nebulising membrane in which liquid is vaporised or nebulised by high-frequency vibration of the membrane.

The electronic control apparatus, for example, is a microcontroller or a microprocessor. Generally, such electronic control apparatuses are usually used to perform computing processes of any kind. Due to the more compact design and architecture of these components, it is possible for the control apparatuses to be used in small electronic devices. On the one hand, the electronic control apparatus is preferably configured and adapted to calculate the administration data for delivering the active substance from the drug data as a function of an air volume while a patient is taking a breath on the inhaler, on the other hand, the electronic control apparatus is capable of a large number of further computing and work processes, in particular it can be used for operating and analysing the inhaler. The electronic control apparatus is more preferably configured and adapted to operate the inhaler with the operating data in order to ensure basic functionality. In addition, the electronic control apparatus preferably uses appropriate sensors to monitor the delivery quantity or the active substance composition while a breath is being taken. Alternatively or additionally, the electronic control apparatus uses appropriate sensors to monitor the volume of air inhaled through the inhaler during a puff, making it possible to calculate specific quantities of the active substance delivered. In addition, the electronic control apparatus advantageously comprises software or a program for operating the electronic control apparatus, in particular for controlling the inhaler and for editing and processing data.

The active substance delivery data detected by the sensor system of the flow measuring apparatus can be determined using the inhaler according to the invention, as a result of which concrete conclusions about the quantities of active substance administered can be obtained when compared with the active substance administration data. For example, comparison of the data is conceivable in relative numbers of active substance delivery data and active substance administration data. The comparison should also include the determination of absolute numbers of the active substance administration data and active substance delivery data, i.e. a determination of the individual values, in order to compare them with each other only afterwards if necessary.

According to the invention, the active substance delivery data is determined for part or the entire duration of an inhalation process. Subsequently, the active substance delivery data determined by means of the flow measuring apparatus are compared with the active substance administration data stored in the data storage device. According to the invention, the predetermined puff profile is compared with measured values which are recorded during a specific inhalation puff.

In particular, the invention creates data based on the air flow measured, i.e. the air flowing in the air channel and/or the active substance flowing through the air channel, whereby the air flowing in the air channel usually includes or picks up the flowing active substance. In this way, the quantity of active substance delivered during the inhalation process is preferably detected during an inhalation process, from which the active substance delivery data are determined. Due to the preferred data acquisition between the air flow during an inhalation process, and preferably also the quantity of active substance delivered during the inhalation process, there is comprehensible data available for the actual active substance delivery data.

Various reactions can take place based on the result of comparing the active substance delivery data and the active substance administration data. In particular, based on the comparison, it is possible to extensively control or adjust the active substance administration data or the administration data in order to bring about complete absorption of the active substance by the user in subsequent inhalation processes.

Advantageously, the sensor system with the flow measuring apparatus can be a differential pressure measuring apparatus which enables reliable measurement of the air volume flow (or air mass flow) in a mobile inhaler using simple means. In this embodiment, the differential pressure measuring apparatus comprises a first pressure sensor, which is arranged to measure the ambient air pressure of the inhaler, and a second pressure sensor, which is arranged to measure the air pressure in the air channel of the inhaler. By knowing the cross-section of the air channel at the measuring location of the second pressure sensor and the pressure difference measured between the first and second pressure sensor, it is possible to determine the air volume flow through the inhaler.

In another embodiment, the flow measuring apparatus can advantageously be a hot-wire measuring apparatus (thermal anemometer). In this case, at least one wire element arranged in the air channel of the inhaler is electrically heated. As a result of the flow around the wire element, heat is transported into the flowing air, i.e. the wire element is cooled. By measuring the electrical resistance, which depends on the temperature, it is possible to determine the flow speed, and thus the volume flow, of the air flowing through the air channel.

It is also possible by means of the flow measuring apparatus to determine the air volume flow over time. Advantageously, this results in a puff profile or measured puff profile of the entire puff or inhalation process.

Preferably, the sensor system comprises a liquid quantity sensor for detecting the quantity of liquid delivered by a vaporising apparatus during an inhalation puff. This allows the quantity of liquid to be vaporised to be adjusted more accurately to the respective puff profile of the user.

In an advantageous embodiment, the liquid quantity sensor is an air humidity sensor. The air humidity sensor preferably comprises two humidity measuring elements which are arranged in the air channel of the inhaler for measuring the air humidity. The first humidity measuring element is arranged in the region of the air inlet or in the air channel upstream of the administration element and measures a reference value of the air humidity before liquid vapour is added. The second humidity measuring element is arranged in the air channel downstream of the administration element, for example in the region of the mouthpiece, and measures the air humidity directly before the user inhales the air-aerosol-vapour mixture.

With a known quantity of water in the liquid to be vaporised and calibration of the second humidity measuring element, the vaporised quantity of liquid can be determined from the increase in air humidity, i.e. the difference between the measured values from the second and first humidity measuring element, and can be taken into account in the further process.

In a preferred embodiment, the inhaler comprises at least one energy source, it being possible, on the one hand, to use the at least one energy source to generate the inhalable active substance, for example for a nebulising or heating element, and, on the other hand, it being provided for supplying energy to the further electrical and electronic components of the inhaler.

The inhaler preferably comprises a communication device configured and adapted to transmit and/or receive data. The communication device represents a device for data transmission for exchanging data with the inhaler, or for transmitting and/or receiving data, in other words, an exchange of data is possible by means of the communication device. Thus, it is possible by means of the communication device for the inhaler to connect to further communication devices in order to transmit and/or receive data from them. The communication device according to the invention is preferably configured and adapted to transmit and/or receive data based on any protocol. Particularly preferably, the data are transmitted wirelessly (e.g. based on Bluetooth, WLAN, ZigBee, NFC, RFID, Wibree, WiMAX, mobile radio, optically, etc.), alternatively, wired data transmission is also possible. The communication device can be used to transmit data from an external reference source to, for example, the electrical control apparatus and/or to the electronic data storage device.

A particularly preferred embodiment is characterised in that data can be provided to the electronic data storage device by means of the communication device and/or by means of a storage unit comprising the active substance container. The data are preferably used to operate the inhaler, i.e. for example, to authenticate the product or the user, to start up the inhaler, to install or update the firmware, etc. and/or to provide information about the active substance to be inhaled by the user, i.e. for example, information about the type of active substance, the active substance concentration, the carrier medium and the regimen. The information contains in particular the active substance administration data for the inhaler to administer the active substance or the actual administration data for operating the inhaler. The data may be standardised and allow control of the administration element to enable the inhaler to be used as intended. Provision of the data by means of the communication device and/or by means of the active substance container ensure that data are provided for operating the inhaler via suitable supply channels. In advantageous embodiments, data, in particular the active substance administration data, are provided to the electronic data storage device only by means of the communication device and/or by means of a storage unit comprising the active substance container. This prevents incorrect operation or incorrect provision of active substance administration data.

An advantageous development is characterised in that the communication device is configured and adapted to communicate with an external deployment source containing the active substance administration data in order to receive the active substance administration data and/or to transmit data. The data to be transmitted are in particular the active substance delivery data detected by means of the flow measuring apparatus. Communication with a deployment source enables direct transmission of the active substance administration data to the inhaler. The external deployment source is used in particular by physicians, hospitals, pharmacies and other qualified authorised persons to preferably establish an encrypted data connection to the communication device of the inhaler. Advantageously, for this purpose the relevant authorised persons define a plan (regimen) for delivering the active substance by the inhaler and transmit it as active substance administration data (a code) to the inhaler by means of a secure data connection. The communication device of the inhaler receives the active substance administration data (the code) and stores it in the electronic data storage device. This provides a safe and reliable device for providing the data for delivering the active substance by the inhaler via a reliable source. Preferably, the data, in particular the active substance administration data, can only be transmitted to the inhaler via the external deployment source. Particularly preferably, prior authentication or authorisation of the authorised person is required (for example via the digital physician ID card) in order to transmit data to the inhaler and/or receive data from the inhaler. In this case, different user groups of the authorised person can have different rights with regard to the availability or modification of the data, for example concerning the regimen or the active substance.

A particularly preferred embodiment is characterised in that the external deployment source comprises an application terminal and/or cloud storage and/or a mobile application in order to receive the active substance administration data by means of the communication device and/or to transmit data. The data to be transmitted are in particular the active substance delivery data detected by means of the flow measuring device. More preferably, the external deployment source comprises or uses means for communicating with the communication device of the inhaler, in particular based on Bluetooth, WLAN, ZigBee, NFC, RFID, Wibree, WiMAX, mobile radio, optically, etc., particularly preferably an encrypted data connection.

In a further advantageous embodiment of the invention, the inhaler can be linked to a mobile application of a communication terminal by means of the communication device in order to transmit and/or receive data, it being possible to receive the active substance administration data from the cloud storage and/or from the application terminal via the mobile application and it being possible to transfer them to the electronic data storage device of the inhaler. This enables location-independent transmission of the data from or to the inhaler, in particular of the active substance administration data or the administration data.

An expedient embodiment of the invention is characterised in that the inhaler comprises at least one motion sensor to detect specific movements of the inhaler before, during and/or after delivery of the active substance. In this way, it is possible, on the one hand, to draw conclusions on the type of use or position of the inhaler during the inhalation process which can be taken into account for determining the active substance delivery data and, on the other hand, it is possible to carry out actions via the motion sensor, for example control of the inhaler using data of the at least one motion sensor.

According to a further preferred embodiment of the invention, the inhaler further comprises a blocking element, the blocking element in particular disabling the administration element to block the delivery of the active substance from the active substance container. In further advantageous embodiments, the inhaler can have a plurality of blocking elements to each carry out specific blocking of components of the inhaler. The blocking element comprises in particular mechanical means, electronic components or software solutions to ensure that there is no unwanted administration of active substance by the administration element. For example, the blocking element actively disables the administration element with a latch, a switch or other physical means; alternatively, software control is provided by means of the electronic control apparatus to prevent actuation of the administration element, thereby preventing delivery of the active substance. A further expedient embodiment of the invention is characterised in that the blocking element can be controlled by means of the electronic control apparatus, the blocking element disabling the administration element if no active substance administration data are available to the electronic control apparatus. The blocking element can prevent misuse or incorrect use of the inhaler, as it is not possible to use the inhaler manually without predetermined active substance administration data. This increases safety for the user and makes it easier to verify the therapy or administration of the active substance.

A preferred development of the invention is characterised in that when the active substance delivery data at least substantially match the active substance administration data, the electronic control apparatus includes one or a plurality of the following actions: Emitting a signal to a signalling device comprising the inhaler and/or to the mobile application and/or to the application terminal and/or to the cloud storage; blocking of the administration element by the blocking element; storing of the at least substantial match of the active substance delivery data with the active substance administration data with a place and/or time stamp on the electronic data storage device and/or on the application terminal and/or in the cloud storage and/or in the mobile application. In this way, it is possible for signals about the inhalation process to be provided to one or a plurality of devices linked to the inhaler, in particular for the active substance administered to the user by the inhaler to substantially match the active substance to be administered. In particular, the electronic control apparatus performs the respective actions. Within the meaning of the invention, “at least substantially” means that the active substance delivery data exactly match the active substance administration data, or slight deviations exist between active substance delivery data and active substance administration data, i.e. deviations ranging from 1% to 10%. The signal is thus emitted based on the result of the comparison between the active substance delivery data detected by means of the flow measuring apparatus and the active substance administration data. Preferably, a signal is emitted after each of the inhalation processes to provide continuous information about the quality of the inhalation process. For example, the signal can include a visual, acoustic, electronic and/or mechanical emission. In particular, the aim of the signal emitted is to inform the user that the inhalation process was successful and the inhalation process has ended. For example, the signals can be sounds, push messages, e.g. on a mobile application, LED displays, vibrations, etc., it being possible to transmit the respective signals on the inhaler and/or on the mobile application and/or on the application terminal and/or on the cloud storage. More preferably, signals can be emitted when the inhaler is ready for operation and/or when the inhalation process can be carried out.

The at least substantial matching is compared by monitoring and measuring the air volume inhaled by the user during the inhalation process, thus usually during a puff, using the appropriate sensor system in the inhaler. In this case, the microcontroller compares the measured air volume with the target value of the air volume, or with the quantity of active substance contained therein. In other words, the electronic control apparatus checks whether the desired delivery of active substance into the air flow was successful, i.e. whether the active substance delivery data correspond to the active substance administration data. For this purpose, the measured air volume is compared with the target value of the air volume (“air volume comparison”) within a target interval. If the active substance delivery matches each other in an air volume comparison, the application has also been successful, or the active substance delivery data correspond to the active substance administration data. Preferably, the electronic control apparatus executes at least one of the following reactions (signals) in the event of an at least substantial match between the active substance administration data and the active substance delivery data: The success of the application is indicated to the user, for example by operating a display, green LED, vibration motor, sound, etc.; the data relating to successful use of the inhaler are stored in the data storage device.

An appropriate action can also be blocking of the inhaler by means of the blocking element. This is expedient, for example, if the regimen prescribes a certain pause between inhalation processes, which is stored in the active substance administration data. After this pause, the blocking element is deactivated again and the user can perform a new inhalation process. This prevents misuse or incorrect use of the inhaler. Storing the at least substantial match between the active substance delivery data and the active substance administration data with a place and/or time stamp also ensures that proof can be provided, in particular for the authorised persons (e.g. physicians), of when and/or where the user took the active substance.

A further expedient configuration of the invention is characterised in that, in the case of a defined deviation of the active substance delivery data from the active substance administration data, the electronic control apparatus includes one or a plurality of the following actions: Emitting a signal to a signalling device comprising the inhaler and/or to the mobile application and/or to the application terminal and/or to the cloud storage; adjusting the administration data for delivering the quantity of the active substance for mixing with the air flowing in the air channel; blocking of the administration element by the blocking element; receiving new active substance administration data by means of the communication device for generating new administration data; storing of the defined deviation of the active substance delivery data from the active substance administration data with a place and/or time stamp on the electronic data storage device and/or on the application terminal and/or in the cloud storage and/or in the mobile application. Due to the control according to the invention, the inhaler adapts to a user, so to speak, by taking into account the individual inhalation process of a user during delivery of the active substance or delivery of the fluid into the air flow. In this way, it is possible for signals about the inhalation process to be provided to one or a plurality of devices linked to the inhaler, in particular that a defined deviation of the active substance delivery data from the active substance administration data exists, i.e. if the quantity of active substance delivered by the inhaler does not correspond to the quantity of active substance taken by the user. There is thus an opportunity to provide monitoring of the quantity of active substance actually delivered or taken, which means that appropriate actions can then be initiated if there is a lack of conformity. The aim of the actions initiated is to monitor the quantity of active substance delivered or taken, which means that in the next step the quantity of active substance to be delivered can be adjusted so that in future the quantity of active substance delivered at least substantially corresponds to the quantity of active substance taken by the user, or so that the active substance delivery data at least substantially match the active substance administration data.

Preferably, a signal is emitted after each of the inhalation processes to provide continuous data/information about the quality of the inhalation process. For example, the signal can include a visual, acoustic, electronic and/or mechanical emission. In particular, the aim of the signal emitted is to inform the patient whether the inhalation process was successful and whether the inhalation process has ended. For example, the signals can be sounds, push messages, e.g. on a mobile application, LED displays, vibrations, etc., it being possible to transmit the respective signals to the inhaler and/or to the mobile application and/or to the application terminal and/or to the cloud storage. More preferably, signals can be emitted when the inhaler is ready for operation and/or when the inhalation process can be carried out.

The defined deviation of the active substance delivery data from the active substance administration data is compared by monitoring and measuring the air volume inhaled by the user during the inhalation process, thus usually during a puff, using the appropriate sensor system in the inhaler. In this case, the microcontroller compares the measured air volume with the target value of the air volume (“air volume comparison”) within a target interval, or with the quantity of active substance contained therein. In other words, the electronic control apparatus checks whether the desired delivery of active substance into the air flow was successful, i.e. whether the active substance delivery data correspond to the active substance administration data. In this case, the active substance delivery data are calculated based on the measured values of the air flow and are preferably measured values of air volume flow and/or air mass flow measured by the flow measuring apparatus as a function of time and/or pressure changes in the region of the inhaler through which the air mass flow passes during the inhalation process. The active substance administration data form a defined, ideal administration of the active substance, which is verified by the chronological sequence of the air or air/vapour volume flow (and/or the air or air/vapour mass flow) that passes through the inhaler during at least part or the entire ideal inhalation process of the user. In this case, the stored active substance delivery data can be used as calibration for further inhalation processes of the active substance. Preferably, the electronic control apparatus executes at least one of the following reactions (signals) in the event of a defined deviation of the active substance delivery data from the active substance administration data: The success of the application is indicated to the user, for example by operating a display, red LED, vibration motor, sound, etc.; the data relating to use of the inhaler are stored in the data storage device. Alternatively, it is conceivable that the inhaler has a fixed predetermined active substance administration data profile and the user is trained by means of corresponding feedback to adhere to an air flow suitable for this, i.e. to an inhalation profile, by appropriately indicating deviations to the user.

More preferably, an appropriate action includes adjusting the activation of the administration element, for example, an administration element configured as a heating element (more accurately the heating current flowing through the heating element and/or the heating duration and/or a pulse-pause ratio of the activation), adjusting the vibration speed of a piezoelectric element or a nebulising membrane or adjusting the exit speed of a compressed gas from a nozzle by setting a gas pressure, e.g. of the air pressure for the quantity of liquid to be administered, and/or indicating corresponding information for a user on a signalling device.

A preferred option is to indicate to the user of the inhaler by means of an appropriate signalling device that, for example, the quantity of active substance delivered during the inhalation process was insufficient. In the scenario of an excessive inhalation process and a possible overdose associated therewith, administration can be stopped with the help of the electronic control system so that it is possible to avoid the occurrence of adverse drug reactions or, in an emergency, appropriate measures (e.g. calling the emergency physician or ambulance) can be initiated. When using the inhaler or the active substance administered therewith, it is also possible to preferably track adverse reactions directly with the help of an app. When the data of many users are viewed together, it may be possible to develop an application profile/dosage with which adverse reactions can be reduced.

By adjusting the administration data, it is possible to modify the modalities of administering the active substance which also results in altered absorption of the active substance by the user. In particular, the administration is altered to the effect that the duration of the inhalation process is varied, for example to divide the quantity of active substance to be administered into two inhalation processes in the future. The process can be dynamic and is modified depending on the active substance administration data. For example, if the measured value or a value calculated from the measured values deviates from a target value, for example the ratio (total prescribed quantity of the active substance composition)/(total quantity of air inhaled during the breath), the electronic control apparatus can actuate the administration element in such a way that the target value is reached. Once the target value for the total quantity of active substance delivered has been reached, the electronic control apparatus can switch off the administration element, even if the inhalation process of the patient is still continuing. Once the inhalation process has ended, the electronic control apparatus performs an action depending on the success of an inhalation process (for example, indication to the user that the active substance has been successfully administered or that it is necessary to repeat an inhalation process).

An appropriate action can also be blocking of the inhaler by means of the blocking element. This is expedient, for example, if the values determined appear to make it necessary for a consultation by a authorised person, e.g. if the active substance delivery data deviate from the active substance administration data by more than 50%. However, this can also depend in particular on the active substance and/or be stored in the active substance administration data.

Receiving new active substance administration data by means of the communication device for generating new administration data provides the authorised person with the opportunity to adjust the active substance administration data to the user. This may preferably then be necessary if the active substance delivery data clearly deviate, i.e. with deviations exceeding 50% for example, from the active substance administration data. However, this can also depend in particular on the active substance and/or be stored in the active substance administration data.

More preferably, the quantity of the active substance delivered is stored in absolute numbers in order to provide the most comprehensive documentation possible of the relevant key figures. In this way, the values can be accessed quickly and easily if required which facilitates further actions.

Storing the at least substantial match between the active substance delivery data and the active substance administration data with a place and/or time stamp also ensures that, if necessary, proof can be provided, in particular for the authorised persons, for example physicians, of when and/or where the user took the active substance. The stored data include in particular unsuccessful use of the inhaler with identification of the failure, the cumulative quantity of active substance presumably delivered and the data of a second, subsequent attempt, if any.

Stored data can preferably be transmitted to an electronic mobile device, e.g. a smartphone, laptop, smartwatch or tablet PC, with the help of a communication device. In this case, users themselves can monitor use of the inhaler with the help of the mobile device. They can also set up reminders on the mobile device if the inhaler is to be used at certain times or at recurring intervals.

Advantageously, in clinical studies from phase I onwards, for example, regular use and thus complete documentation of inhaler use can be ensured. Also, when the data are transmitted to an electronic mobile device, users can note their personal perceptions before, after and/or during use of the inhaler. In this way, for example, a causal relationship between certain physical reactions and use of the inhaler or the active substance applied therewith can be closely documented. The information thus obtained, e.g. on the tolerance of an active substance, can also be relevant for the physician, for example, when treating an illness with an active substance that is already approved.

More preferably, the inhaler enables long-term monitoring of the respective user. Thus, monitoring with regard to the frequency of use is also possible. Increasing frequency of use can be a sign of habituation or an insufficient quantity of active substance per inhalation. Furthermore, in this way it is possible to identify dependence on/addiction to the active substance. For this purpose, the active substance delivery data or active substance administration data can be stored on the electronic data storage device and/or a storage unit of the active substance container and/or on the application terminal and/or in the cloud storage and/or in the mobile application or on the communication terminal. In this way, actual use of the inhaler and application of the active substance can be tracked and linked to a physical change in the user. More preferably, authorised persons can verify correct application of the active substance or active substance administration data at any time when the data are accessed remotely, e.g. cloud storage and/or application terminal. In a further advantageous configuration of the invention, users can actively and/or passively automatically provide information about their physical condition and physical parameters before, during and/or after using the inhaler or the inhalation process, e.g. with the help of a mobile application. In further advantageous embodiments, it is possible to store data of further devices which have access to physiological data, e.g. blood pressure, pulse, temperature, oxygen saturation, fitness, weight, blood sugar, etc., with the data from the last use of the inhaler.

The object is also achieved by a method for administering an active substance with an inhaler, comprising a housing having a main body, the main body being configured and adapted to receive an active substance container containing an active substance, an air channel extending within the housing between at least one air inlet opening and one inhalation opening, an administration element for nebulising or vaporising the active substance fed from the active substance container to the administration element for mixing with air flowing in the air channel, an electronic control apparatus, an electronic data storage device and a sensor system having a flow measuring apparatus for measuring the volume flow and/or mass flow of the air flowing through the air channel and/or of the active substance flowing through the air channel for storage as active substance delivery data in the electronic data storage device, comprising the steps: Providing active substance administration data in the electronic data storage device of the inhaler, converting the active substance administration data into administration data by means of the electronic control apparatus of the inhaler, controlling the mixing of the active substance, by the administration element, with the air flowing in the air channel using the administration data, calculating the active substance delivered from the active substance container based on the active substance delivery data detected by means of the flow measuring apparatus, comparing the active substance administration data with the active substance delivery data.

To avoid repetition, reference is made in connection with the method according to the invention to the merits already described in detail in connection with the inhaler according to the invention. They also apply by analogy to the method according to the invention given below.

A development of the method is characterised in that the inhaler further comprises a communication device in order to transmit and/or receive data.

An expedient embodiment of the invention is characterised in that data is provided to the electronic data storage device by means of the communication device and/or by means of a storage unit comprising the active substance container.

According to a further preferred embodiment of the invention, the communication device communicates with an external deployment source which contains the active substance administration data in order to receive the active substance administration data and/or to transmit data.

A further expedient configuration of the invention is characterised in that the inhaler is linked to a mobile application of a communication terminal by means of the communication device before transmitting/receiving data for the first time.

A development is characterised in that the active substance administration data are received from the cloud storage of the inhaler and/or from the application terminal and/or via the mobile application and are transferred to the electronic data storage device, and in particular are transmitted to the communication device of the inhaler by means of the mobile application.

In a further advantageous embodiment of the invention, data are transmitted by means of the inhaler to the mobile application and/or the application terminal and/or the cloud storage.

In an advantageous embodiment of the invention, when the active substance delivery data at least substantially match the active substance administration data, one or a plurality of the following actions are performed by means of the electronic control apparatus: Emitting a signal to a signalling device comprising the inhaler and/or to the mobile application and/or to the application terminal and/or to the cloud storage, blocking of the administration element by the blocking element, storing of the at least substantial match of the active substance delivery data with the active substance administration data with a place and/or time stamp on the electronic data storage device and/or on the application terminal and/or in the cloud storage and/or in the mobile application.

A further expedient configuration of the invention is characterised in that, in the case of a defined deviation of the active substance delivery data from the active substance administration data, one or a plurality of the following actions are performed by the electronic control apparatus: Emitting a signal to a signalling device comprising the inhaler and/or to the mobile application and/or to the application terminal and/or to the cloud storage, adjusting the administration data for delivering the quantity of the active substance for mixing with the air flowing in the air channel, blocking of the administration element by the blocking element, receiving new active substance administration data by means of the communication device for generating new administration data; storing of the defined deviation of the active substance delivery data from the active substance administration data with a place and/or time stamp on the electronic data storage device and/or on the application terminal and/or in the cloud storage and/or in the mobile application.

Particularly preferably, the method is carried out with an inhaler as described herein. The advantages and effects arising from this have already been described in connection with the inhaler, which is why reference will be made to the preceding passages to avoid repetitions.

The advantages arising from the particular method steps have already been described in connection with the inhaler and the arrangement, which is why reference will be made to the relevant passages to avoid repetitions.

In an advantageous embodiment of the invention, a new inhaler or active substance container can be ordered automatically when the quantity of active substance in the inhaler or in the active substance container falls below a certain level or when a specified limit of an accumulated active substance is exceeded.

Preferably, the inhaler can be used stand-alone or, for example, in conjunction with a mobile application to remind the user to use the inhaler regularly. The inhaler preferably has a real-time clock (RTC) which, after initial synchronisation with a clock in, for example, a communication terminal, activates signalling devices in the inhaler, e.g. LED display, speaker and/or vibration device, to indicate the start of use of the inhaler. Alternatively and/or additionally, a time restriction on using the inhaler can be specified. After determining a successful delivery of the active substance, it is possible to disable use of the inhaler for a certain time, preferably by means of the blocking element. Disabling may be outside the specified periods of use of the inhaler. For example, treatment is carried out in the mornings from 7:00-9:00, at midday from 12:00-14:00, and in the evenings from 19:00-21:00. The inhaler cannot be activated and used outside these times. Furthermore, disabling can provide prevention against addiction, limiting, for example, a maximum number of uses of the inhaler, for example when used for painkillers.

The object is also achieved by an arrangement of the type referred to hereinbefore in that the inhaler is configured and adapted as described herein and the communication terminal and/or the application terminal and/or the cloud storage are configured and adapted for transmitting and/or receiving data with the inhaler.

To avoid repetition, reference is made in connection with the arrangement according to the invention to the merits already described in detail in connection with the inhaler according to the invention. They also apply by analogy to the arrangement according to the invention.

Particularly advantageously, the method is carried out with an inhaler as described herein and/or with an arrangement as described herein.

### BRIEF DESCRIPTION OF THE DRAWINGS

Further expedient and/or advantageous features and developments of the inhaler as well as the arrangement and the method emerge from the dependent claims and the description. Particularly preferred embodiments are explained in greater detail with reference to the attached drawings. The drawings show:

FIG. 1 a schematic view of an inhaler according to the invention in longitudinal section;

FIG. 2 a schematic illustration of the inhaler in conjunction with an external deployment source;

FIG. 3 a flow chart for use of an inhaler;

FIG. 4 a flow chart for successful use of an inhaler;

FIG. 5 a flow chart for unsuccessful use of an inhaler; and

FIG. 6 a schematic illustration of the use of an arrangement according to the invention.

### DETAILED DESCRIPTION OF THE INVENTION

The inhaler according to the invention, the method according to the invention and the arrangement according to the invention are described with reference to the aforementioned figures. To avoid repetition, the statements made about the inhaler also apply to the method according to the invention and to the arrangement according to the invention, so that statements are made below only on selected aspects of the method according to the invention and the arrangement according to the invention, detached from the inhaler according to the invention.

The inhaler shown in the drawing is configured and adapted for administering an active substance. The invention relates in the same way to comparable products for administering active substances with which other substances, for example luxury goods/stimulant substances or medical preparations, are to be administered. In particular, this explicitly includes electronic cigarette products and medical inhalers which have a similar construction.

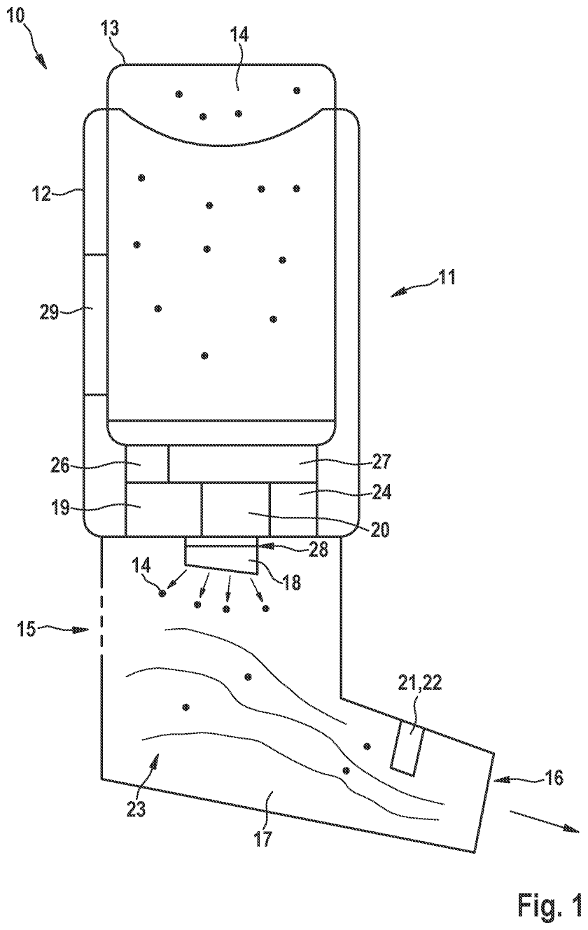

The inhaler 10 shown in FIG. 1, comprises a housing 11 having a main body 12, the main body 12 being configured and adapted to receive an active substance container 13 containing an active substance 14, an air channel 17 extending between at least one air inlet opening 15 and an inhalation opening 16 within the housing 11, an administration element 18 for nebulising or vaporising the active substance 14 supplied to the administration element 18 from the active substance container 13 for mixing with air 23 flowing in the air channel 17, an electronic control apparatus 19, an electronic data storage device 20 and a sensor system 21 having a flow measuring apparatus 22 for measuring the volume flow and/or mass flow of the air 23 flowing through the air channel 17 and/or of the active substance 14 flowing through the air channel 17 for storage as active substance delivery data in the electronic data storage device 20. The inhaler 10 preferably comprises at least one energy source 27, for example for supplying the electronic control apparatus 19 as well as the administration element 18 and the further electrical or electronic components with energy.

This inhaler 10 is characterised according to the invention in that the electronic control apparatus 19 of the inhaler 10 is configured and adapted to convert active substance administration data, provided in the electronic data storage device 20, for administering the active substance 14 by the inhaler 10 into administration data in order to control mixing of the active substance 14, by the administration element 18, with air 23 flowing in the air channel 17, and the electronic control apparatus 19 being adapted to calculate the active substance 14 delivered from the active substance container 13 based on the active substance delivery data detected by means of the flow measuring device 22 and to compare same with the active substance administration data.

The air 23 flowing in the air channel 17 is shown in stylised form in FIG. 1, whereby the air 23 flowing in the air channel 17 is not usually visible, or the flowing air 23 is regularly formed, for example, as vapour or aerosol. The air 23 flowing in the air channel 17 includes the active substance 14 during use of the inhaler 10, i.e. during the inhalation process, provided that the user sucks on the inhalation opening 16. In the process, the active substance 14 is transported by the air 23 flowing in the air channel 17.

The housing 11 of the inhaler 10 can basically have any colours and shapes and is not limited in design and choice of material. In this respect, the housing 11 is structured so that it includes a region for receiving and/or enclosing the active substance container 13 within the housing 11 of the inhaler 10. The inhaler 10 shown in FIG. 1 corresponds to the schematic construction of a medical inhaler, although a more elongated construction, comparable to that of an electronic cigarette, for example, is also conceivable. For the inhaler 10 according to the invention, it is not relevant whether the inhaler 10 or parts thereof are configured as disposable or reusable items. The active substance container 13 can preferably be configured as a disposable item and can have a further electronic data storage device/a storage unit, not shown in the figures, on which, for example, active substance administration data and/or administration data for controlling the inhaler 10 are stored.

The active substance 14 to be administered, which is located in the active substance container 13, is preferably dissolved in a liquid and is present as a mixture. Furthermore, the active substance 14 can be present in the active substance container 13 as a gas, or can be a compressed liquid or can be dissolved in a compressed liquid. If the active substance 14 is dissolved in a liquid, the mixture comprises, for example, one or a plurality of the following constituents: 1,2-propylene glycol, glycerin, water, at least one flavouring, and one or a plurality of active substances 14. The composition of the liquid, however, depends on the active substance 14 to be administered and can be designed according to the pharmacological requirements.

The schematically illustrated inhaler 10 serves the purpose of administering the active substance 14 via the inhalation opening 16 to the respective user of the inhaler 10, who is not shown in the drawings. For this purpose, the active substance 14 is present in the active substance container 13 and is administered by means of the administration element 18 to air 23 flowing in the air channel 17, as a result of which this air 23 is in turn orally ingested by the user. The administration element 18 can be configured as required depending on the active substance 14 to be administered, whereby in each case administration of the active substance 14 from the active substance container 13 is provided for. The administration element 18 is controlled by means of the electronic control apparatus 19 in order to administer the active substance 14 in accordance with the active substance administration data provided. The active substance administration data are provided in the electronic data storage device 20 and can be converted into administration data by means of the electronic control apparatus 19. The administration data form the data for administering the active substance 14 by means of the administration element 18 which is controlled by means of the electronic control apparatus 19. The active substance 14 generated by the administration element 18 in vapour or aerosol form escapes from the administration element 18 and is mixed with the air 23 flowing in the air channel 17, see FIG. 1. The flow measuring apparatus 22 uses the sensor system 21 to determine the quantity of active substance 14 administered and uses the electronic control apparatus 19 to calculate the active substance delivery data in order to compare them with the active substance administration data.

The electronic data storage device 20 is advantageously non-volatile and is used, for example, to store the active substance administration data, the administration data, the active substance delivery data, the data of the sensor system and further general information or parameters about operation of the inhaler 10. The data storage device 20 can be part of the electronic control apparatus 19. Advantageously, the data storage device 20 stores information on the composition of the liquid or active substance 14 stored in the active substance container 13, information on the operating principle of the administration element 18, in particular on power/temperature control, etc., data on condition monitoring or system testing, for example leak testing, data relating to copy protection and counterfeit protection, an ID for unique identification, serial number, date of manufacture, expiry date, number of inhalation processes and/or usage time.

The sensor system 21 comprises a flow measuring apparatus 22 which is configured here as a differential pressure measuring apparatus. For this purpose, the flow measuring apparatus 22 comprises a first pressure sensor, not shown in detail in the figures, which is arranged such that it is adapted for measuring the atmospheric air pressure outside the housing 11 of the inhaler 10. For example, a measuring opening in the housing 11 can be provided to connect the first pressure sensor to the atmosphere in a flow-conducting manner. The flow measuring apparatus 22 further comprises a second pressure sensor, not shown in detail in the figures, which is arranged such that it is adapted for measuring the air pressure prevailing in the air channel 17. By knowing the cross-section of the air channel 17 at the measuring location of the second pressure sensor and the pressure difference measured between the first and second pressure sensor, the electronic control apparatus 19 can calculate the air volume flow through the inhaler 10 and, by repeatedly measuring, the air flow volume over time.

The sensor system 21 can have a vapour quantity sensor to detect the quantity of active substance, liquid or vapour administered by the administration element 18 during an inhalation process. The vapour quantity sensor, for example, can be an air humidity sensor, not shown in detail.

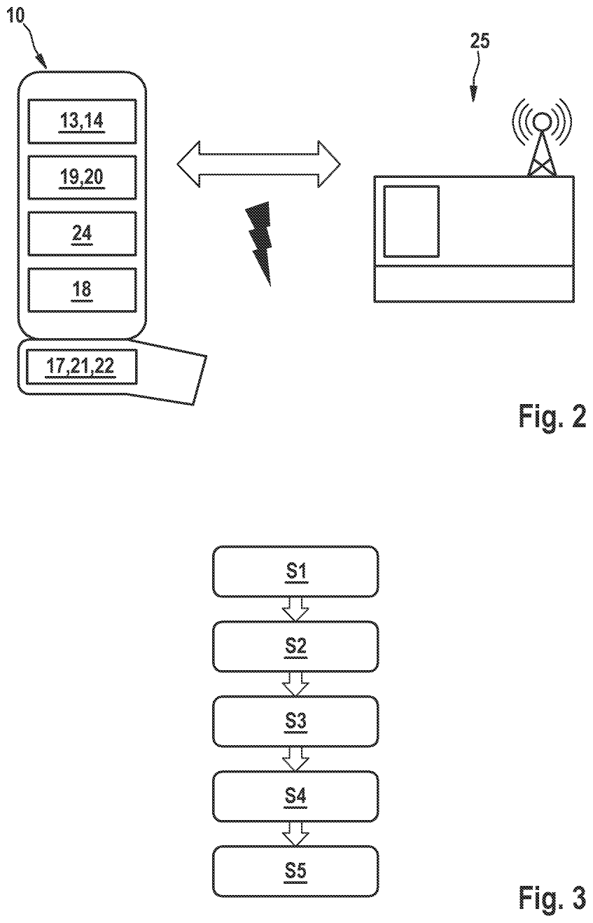

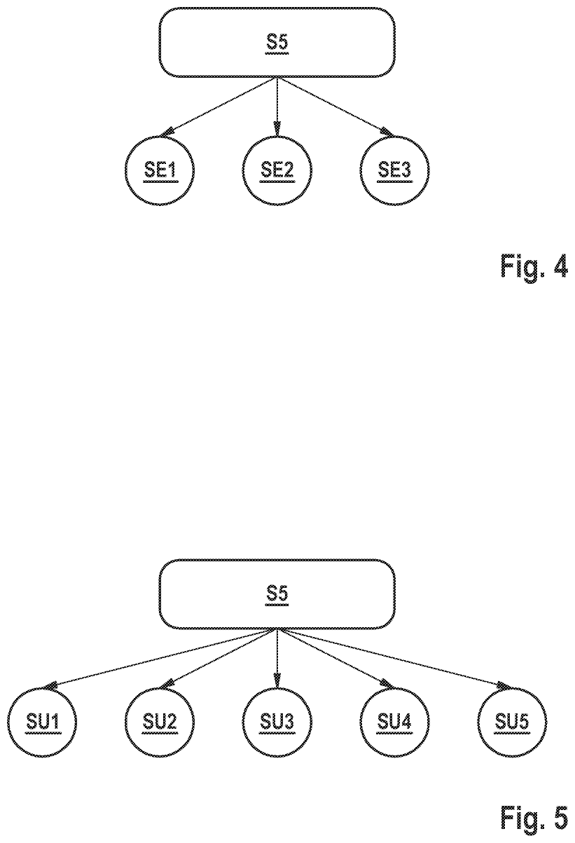

The inhaler 10 preferably comprises a communication device 24, configured and adapted to transmit and/or receive data. Preferably, data can be provided to the electronic data storage device 20 by means of the communication device 24 and/or by means of a storage unit, not shown in the figures, which comprises the active substance container 13. In this case, the data are determined in particular for operating the inhaler 10 or for administering the active substance 14 and/or data during the inhalation process. The communication device 24 is configured and adapted to communicate with an external deployment source 25 (see FIG. 2) which contains the active substance administration data in order to receive the active substance administration data and/or to transmit data. The transmitting and receiving of data is indicated in FIG. 2 by the double arrow between inhaler 10 and the deployment source 25. The inhaler 10 and the deployment source 25 are shown in simplified schematic form, with the ability to communicate with each other apparent from FIG. 2. The communication device 24 of the inhaler 10 preferably comprises means for wirelessly transmitting data, e.g. based on Bluetooth, WLAN, ZigBee, NFC, RFID, Wibree, WiMAX, mobile radio, optically; alternatively, wired data transmission or communication is also possible. The external deployment source 25 preferably also comprises at least one of the aforementioned means or communication options to configure and adapt a connection or communication with the inhaler 10.

Preferably, the external deployment source 25 (see FIG. 2) comprises an application terminal and/or cloud storage and/or a mobile application, not shown in the figures, in order to receive the active substance administration data by means of the communication device 24 and/or to transmit data. In this case, the external deployment source 25 is selected depending on the application purpose or configuration of the inhaler 10, the active substance 14 to be administered or the relevant authorised person.

Advantageously, the inhaler 10 can be linked by means of the communication device 24 to a mobile application of a communication terminal, shown schematically in FIG. 2 as external deployment source 25, in order to transmit and/or receive data, it being possible to receive the active substance administration data from the cloud storage and/or from the application terminal via the mobile application and to transmit them to the electronic data storage device 20 of the inhaler 10. Transmission of the data is shown schematically in FIG. 2 as a double arrow. The external deployment source 25 can be divided into a plurality of separate components which fulfil different sub-functions of the overall functionality of the external deployment source 25. For example, the mobile application can be used to display supplementary information or to emit signals, while data are being provided in the cloud storage, in order to retrieve them by means of the communication terminal.

Optionally, the inhaler 10 comprises at least one motion sensor 26 to detect specific movements of the inhaler 10 before, during and/or after delivery of the active substance. The motion sensor 26 can be a separate component, or the motion sensor 26 can be integrated into the electronic control apparatus 19, for example.

Preferably, the inhaler 10 further comprises a blocking element 28, the blocking element 28 in particular disabling the administration element 18 to block the delivery of the active substance 14 from the active substance container 13. In FIG. 1, the blocking element 28 is configured as an object, however, electronic control components which stop the administration element 18 from administering the active substance 14 from the active substance container 13 are also conceivable as an alternative. It is additionally possible for the blocking element 28 to be arranged directly at the outlet opening of the active substance container 13 in order to block administration of the active substance 14. The blocking element 28 is preferably controllable by means of the electronic control apparatus 19, the blocking element 28 disabling the administration element 18 if no active substance administration data are available to the electronic control apparatus 19.

The inhaler 10 preferably comprises a signalling device 29. For example, the signalling device 29 comprises visual, acoustic and/or haptic signalling elements, for example one or a plurality of LEDs, a digital display, a display, a haptic signal transmitter and/or an acoustic signal transmitter.

A preferred method is explained below in greater detail with the aid of FIG. 3.

The method is described for administering an active substance 14 with an inhaler 10. The method comprises the steps S1 to S5. In step S1, active substance administration data are provided in an electronic data storage device 20 of the inhaler 10. The data can be provided to the electronic data storage device 20 in a variety of ways. For example, the active substance administration data are provided via a storage unit of the active substance container 13 or can be received by means of a communication device 24.

In step S2, the active substance administration data are converted into administration data by means of an electronic control apparatus 19 of the inhaler 10. The administration data can be used to control administration of the active substance 14 via an administration element 18. The administration data are based on the active substance administration data and convert the data into control signals in order to adjust the active substance administration data to a respective administration element 18.

In step S3, mixing of the active substance 14, by the administration element 18, with air 23 flowing in an air channel 17 is controlled by means of the administration data. The inhaler 10 is activated by the administration data as a function of its comprehensive components, so that the active substance 14 to be administered matches the active substance administration data.

In the following step S4, the active substance 14 delivered from the active substance container 13 is calculated based on the active substance delivery data detected by means of a flow measuring apparatus 22 of the inhaler 10. For this purpose, the air components in particular of the air 23 flowing through the air channel 17 are calculated out during delivery of the active substance and the quantity of the active substance 14 actually administered can be determined and stored in the active substance delivery data. The active substance delivery data thus represent the active substance 14 actually delivered which has been absorbed by the user of the inhaler 10 while carrying out the previous steps S1 to S3.

In step S5, the active substance administration data are compared with the active substance delivery data. Basically, a successful inhalation process is when the active substance delivery data match the active substance administration data. The electronic control apparatus 19 is used to calculate how the inhalation process has gone as a function of the active substance administration data with the active substance delivery data. The comparison can be based on both absolute data and relative data.

The data provided and/or generated while carrying out steps S1 to S5 can be transmitted via a communication device 24 of the inhaler 10 to an external deployment source 25, the external deployment source 25 comprising in particular a mobile application, an application terminal and/or cloud storage.

One or a plurality of the following steps are preferably performed if the comparison carried out in step S5 by means of the electronic control apparatus 19 results in at least a substantial match between the active substance delivery data and the active substance administration data. In step SE1, emitting of a signal to a signalling device 29 comprising the inhaler 10 and/or to the mobile application and/or to the application terminal and/or to the cloud storage. The signal is emitted after the inhalation process has been completed and the comparison of the active substance administration data with the active substance delivery data has ended. In particular, the signal informs the user of the inhaler 10 that the inhalation process has been successfully carried out, i.e. that the active substance administration data match the active substance delivery data. The signal is preferably represented visually, haptically and/or acoustically by means of a signalling device 29. As a result of the signal, further actions concerning the inhaler 10 can be performed by means of the electronic control apparatus 19. In step SE2, for example, blocking of the administration element 18 or the inhaler 10 by the blocking element 28 takes place subsequently or alternatively to SE1. This step is preferably configured to be reversible.

In addition or alternatively to step SE1 or SE2, in step SE3 the at least substantial match of the active substance delivery data with the active substance administration data is stored with a place and/or date stamp on the electronic data storage device 20 and/or on the application terminal and/or in the cloud storage and/or in the mobile application.

One or a plurality of the following steps are preferably performed if the comparison carried out in step S5 by means of the electronic control apparatus 19 results in a defined deviation of the active substance delivery data from the active substance administration data. In step SU1, emitting of a signal to a signalling device 29 comprising the inhaler 10 and/or to the mobile application and/or to the application terminal and/or to the cloud storage. The signal is emitted after the inhalation process has been completed and the comparison of the active substance administration data with the active substance delivery data has ended. In particular, the signal informs the user of the inhaler 10 that the inhalation process has been unsuccessfully carried out, i.e. that the active substance delivery data deviate from the active substance administration data. As a result of the signal, further actions concerning the inhaler 10 can be performed by means of the electronic control apparatus 19, in which case the signal is in particular a control signal.

Additionally or alternatively to step SUL adjusting the administration data for delivering the quantity of the active substance 14 for mixing with the air 23 flowing in the air channel 17 takes place in step SU2. The administration data are carried out based on the comparison between the active substance administration data and the active substance delivery data performed in step S5. In particular, the administration data are adjusted to the effect that delivery of the active substance 14 to air 23 flowing in the air channel 17 is modified by means of the administration element 18. For example, the administration data can be adjusted to the effect that the duration of the delivery of the active substance 14 to air 23 flowing in the air channel 17 is varied by means of the administration element 18; for example, in that the quantity of the active substance 14 to be absorbed previously in an inhalation process is divided over two inhalation processes in order to achieve complete administration of the active substance 14. The administration data are thus adjusted but the active substance administration data are retained. When the active substance administration data are compared with the active substance delivery data, the active substance 14 delivered to the user by the inhaler 10 are compared with each other again.

In step SU3, blocking of the administration element 18 or the inhaler 10 by the blocking element 28 takes place subsequently or alternatively to steps SU1 and/or SU2. This step is preferably configured to be reversible.

In addition and/or alternatively, in step SU4, new active substance administration data are received from the inhaler 10 by means of the communication device 24 for generating new administration data. The new active substance administration data preferably have a modified specification for administering the active substance 14. Preferably, steps SU2 and/or SU4 in particular are repeated as often as desired until there is at least a substantial match between the active substance delivery data and the active substance administration data in the further inhalation processes carried out.

In addition or alternatively to steps SU1 to SU4, in step SU5, the defined deviations of the active substance delivery data from the active substance administration data are stored with a place and/or time stamp on the electronic data storage device 20 and/or on the application terminal and/or in the cloud storage and/or in the mobile application. The process is preferably repeated as often as desired until there is at least a substantial match between the active substance delivery data and the active substance administration data.

Steps SE1 and/or SE2 or steps SU3 and/or SU5 take place if there is an at least substantial match between the active substance delivery data and the active substance administration data after the inhalation process is repeated with modified active substance administration data.

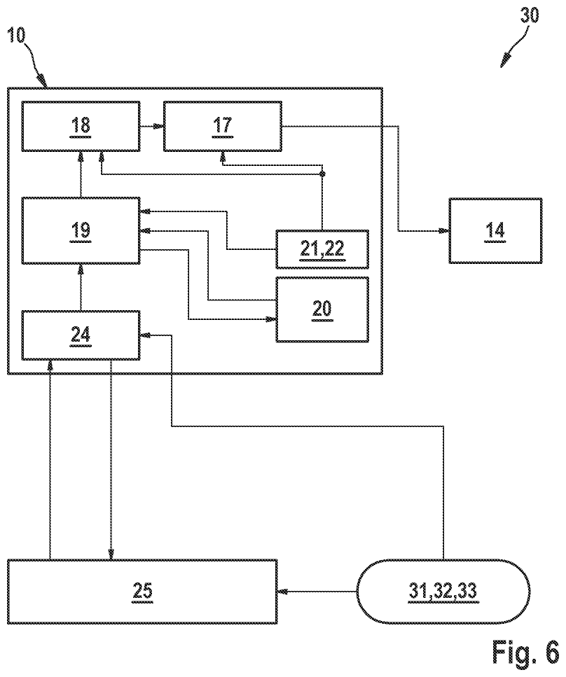

FIG. 6 shows an inhaler 10 preferably as a component of an arrangement 30 which is configured and adapted for administering an active substance 14. The arrangement 30 comprises an inhaler 10 and a communication terminal 32 comprising a mobile application 31 and/or an application terminal and/or a cloud storage 33, the inhaler 10 being configured and adapted according to one or more of claims 1 to 11 and the communication terminal and/or the application terminal and/or the cloud storage being configured and adapted for transmitting and/or receiving data with the inhaler 10.

1-21. (canceled)

22. An inhaler comprising:
a housing having a main body, wherein the main body is configured to receive an active substance container containing an active substance, and an air channel extending within the housing between at least one air inlet opening of the housing and an inhalation opening of the housing;
an administration element for nebulising or vaporising the active substance fed from the active substance container to the administration element for mixing the active substance with air flowing in the air channel;
an electronic control apparatus;
an electronic data storage device; and
a sensor system having a flow measuring apparatus for measuring at least one of a volume flow and a mass flow of at least one of the air flowing through the air channel and the active substance flowing through the air channel for storage as active substance delivery data in the electronic data storage device;
wherein:
the electronic control apparatus is configured to convert active substance administration data, provided in the electronic data storage device for administering the active substance by the inhaler, into administration data in order to control the mixing, by the administration element, of the active substance with the air flowing in the air channel; and
the electronic control apparatus is configured to calculate a quantity of the active substance delivered from the active substance container based on the active substance delivery data detected by means of the flow measuring apparatus and to compare the quantity with the active substance administration data.

23. The inhaler of claim 22, further comprising a communication device configured to either or both transmit or receive data.

24. The inhaler of claim 23, wherein data can be provided to the electronic data storage device by means of at least one of the communication device and a storage unit comprising the active substance container.

25. The inhaler of claim 23, wherein the communication device is configured to communicate with an external deployment source containing the active substance administration data in order to either or both receive the active substance administration data or transmit data.

26. The inhaler of claim 25, wherein the external deployment source comprises at least one of an application terminal, a cloud storage, and a mobile application to either or both receive the active substance administration data by means of the communication device or to transmit data.

27. The inhaler of claim 26, wherein the inhaler is configured to be linked to a mobile application of a communication terminal by means of the communication device to either or both transmit data or receive data, wherein the active substance administration data can be received from at least one of the cloud storage and the application terminal via the mobile application and can be transferred to the electronic data storage device of the inhaler.

28. The inhaler of claim 22, further comprising at least one motion sensor to detect specific movements of the inhaler at least one of before, during and after delivery of the active substance.

29. The inhaler of claim 22, further comprising a blocking element, wherein the blocking element blocks the administration element to disable the delivery of the active substance from the active substance container.

30. The inhaler of claim 29, wherein the blocking element is configured to be controlled by means of the electronic control apparatus, wherein the blocking element is configured to disable the administration element if the active substance administration data is not available to the electronic control apparatus.

31. The inhaler of claim 30, wherein, when the active substance delivery data at least substantially matches the active substance administration data, the electronic control apparatus is configured to take an action including at least one of:
emitting a signal to at least one of a signaling device comprising the inhaler, a mobile application, an application terminal, and a cloud storage;
blocking of the administration element by the blocking element;
storing of an at least substantial match of the active substance delivery data with the active substance administration data with at least one of a place and a time stamp on or in at least one of the electronic data storage device, the application terminal, the cloud storage, and the mobile application.

32. The inhaler of claim 30, wherein, in the case of a defined deviation of the active substance delivery data from the active substance administration data, the electronic control apparatus takes an action including at least one of:
emitting a signal to at least one of a signaling device comprising the inhaler, the mobile application, the application terminal, and the cloud storage;
adjusting the administration data for delivering the quantity of the active substance for mixing with the air flowing in the air channel;
blocking the administration element with the blocking element;
receiving new active substance administration data by means of the communication device for generating new administration data;
storing the defined deviation of the active substance delivery data from the active substance administration data with at least one of a place and time stamp on or in at least one of the electronic data storage device, the application terminal, the cloud storage, and the mobile application.

33. A method for administering an active substance with an inhaler, the inhaler comprising:
a housing having a main body, wherein the main body is configured to receive an active substance container containing an active substance, and an air channel extending within the housing between at least one air inlet opening of the housing and an inhalation opening of the housing;
an administration element for nebulising or vaporising the active substance fed from the active substance container to the administration element for mixing the active substance with air flowing in the air channel;
an electronic control apparatus;
an electronic data storage device; and
a sensor system having a flow measuring apparatus for measuring at least one of a volume flow and a mass flow of at least one of the air flowing through the air channel and the active substance flowing through the air channel for storage as active substance delivery data in the electronic data storage device;
wherein the method comprises:
providing active substance administration data in the electronic data storage device of the inhaler;
converting the active substance administration data into administration data by means of the electronic control apparatus of the inhaler;
controlling the mixing of the active substance, by the administration element, with the air flowing in the air channel using the administration data;
calculating a quantity of the active substance delivered from the active substance container based on the active substance delivery data detected by means of the flow measuring device; and
comparing the active substance administration data with the active substance delivery data.

34. The method of claim 33, wherein the inhaler further comprises a communication device configured to either or both transmit data or receive data.

35. The method of claim 34, wherein data is provided to the electronic data storage device by means of at least one of the communication device and a storage unit comprising the active substance container.

36. The method of claim 35, wherein the communication device communicates with an external deployment source containing the active substance administration data to either or both receive the active substance administration data or transmit data.

37. The method of claim 34, wherein the inhaler is linked to a mobile application of a communication terminal by means of the communication device before a first time of either or both of transmitting data or receiving data.

38. The method of claim 37, wherein the active substance administration data is received from at least one of a cloud storage, an application terminal, and the mobile application by means of the communication device of the inhaler and is transferred to the electronic data storage device of the inhaler.

39. The method of claim 38, wherein data is transmitted to at least one of the mobile application, the application terminal, and the cloud storage by means of the inhaler.

40. The method of claim 38, wherein, when the active substance delivery data at least substantially matches the active substance administration data the electronic control apparatus is configured to take an action including at least one of:
emitting a signal to at least one of a signaling device comprising the inhaler, the mobile application, the application terminal, and the cloud storage;
blocking the administration element with the blocking element;
storing of an at least substantial match of the active substance delivery data with the active substance administration data with at least one of a place and time stamp on or in at least one of the electronic data storage device, the application terminal, the cloud storage, and the mobile application.

41. The method of claim 38, wherein, in the case of a defined deviation of the active substance delivery data from the active substance administration data, the electronic control apparatus is configured to take an action including at least one of:
emitting a signal to at least one of a signaling device comprising the inhaler, the mobile application, the application terminal, and the cloud storage;
adjusting the administration data for delivering the quantity of the active substance for mixing with the air flowing in the air channel;
blocking the administration element with the blocking element;
receiving new active substance administration data by means of the communication device for generating new administration data;
storing the defined deviation of the active substance delivery data from the active substance administration data with at least one of a place and time stamp on or in at least one of the electronic data storage device, the application terminal, the cloud storage, and the mobile application.

42. An arrangement for administering an active substance, the arrangement comprising a communication terminal comprising at least one of a mobile application, an application terminal, and cloud storage, the arrangement further comprising an inhaler comprising:
a housing having a main body, wherein the main body is configured to receive an active substance container containing an active substance, and an air channel extending within the housing between at least one air inlet opening of the housing and an inhalation opening of the housing;
an administration element for nebulising or vaporising the active substance fed from the active substance container to the administration element for mixing the active substance with air flowing in the air channel;
an electronic control apparatus;
an electronic data storage device; and
a sensor system having a flow measuring apparatus for measuring at least one of a volume flow and a mass flow of at least one of the air flowing through the air channel and the active substance flowing through the air channel for storage as active substance delivery data in the electronic data storage device;
wherein:
the electronic control apparatus is configured to convert active substance administration data, provided in the electronic data storage device for administering the active substance by the inhaler, into administration data in order to control the mixing, by the administration element, of the active substance with the air flowing in the air channel;
the electronic control apparatus is configured to calculate a quantity of the active substance delivered from the active substance container based on the active substance delivery data detected by means of the flow measuring apparatus and to compare the quantity with the active substance administration data; and
at least one of the communication terminal, the application terminal, and the cloud storage are configured to either or both transmit data to or receive data from the inhaler.
